(19) United States
(12) Patent Application Publication
(10) Pub. No.: US 2009/0228097 A1
(43) Pub. Date: Sep. 10, 2009

(54) A1 Adenosine Receptor Antagonist-Coated Implantable Medical Device

(75) Inventor: Yunbing Wang, Sunnyvale, CA (US)

Correspondence Address: SQUIRE, SANDERS & DEMPSEY LLP, 1 MARITIME PLAZA, SUITE 300, SAN FRANCISCO, CA, 94111, US

(73) Assignee: Abbott Cardiovascular Systems Inc., Santa Clara, CA (US)

(21) Appl. No.: 12/044,733

(22) Filed: Mar. 7, 2008

(51) Int. Cl. A61F 2/82

(52) U.S. Cl. 623/1.42

(57) ABSTRACT

An implantable medical device with an A1 adenosine receptor antagonist-coating is disclosed.

### FIELD OF THE INVENTION

The present invention is directed to A1 adenosine receptor antagonist-coated implantable medical devices and methods of using for the treatment of disease.

### BACKGROUND OF THE INVENTION

Percutaneous transluminal coronary angioplasty is a procedure for treating heart disease in which a catheter assembly having a balloon portion is introduced percutaneously into the cardiovascular system of a patient via the brachial or femoral artery. The catheter assembly is advanced through the coronary vasculature until the balloon portion is positioned across an occlusive lesion. The balloon is inflated to radially compress against a lesion thereby remodeling the vessel. The balloon is then deflated to allow the catheter to be withdrawn from the patient's vasculature. Restenosis of the artery, however, can develop after the procedure, thereby requiring another angioplasty procedure or a surgical by-pass operation.

Restenosis is thought to involve the body's natural healing process. Angioplasty or other vascular procedures injure vessel walls by removing vascular endothelium, disturbing the tunica intima and causing the death of medial smooth muscle cells. Excessive neoinitimal tissue formation, characterized by smooth muscle cell migration and proliferation to the intima often follows the injury. Proliferation and migration of smooth muscle cells (SMC) from the media layer to the intima can cause an excessive production of extra cellular matrices (ECM), which is believed to be one of the leading contributors to the development of restenosis.

Intravascular stents are sometimes implanted within vessels in an effort to maintain vessel patency by preventing collapse and/or by impeding restenosis. Therapeutic substances coated onto the stents are also often used to further inhibit the development of restenosis. Everolimus, for example, can be used as a coating layer on a stent to decrease restenosis by preventing the proliferation of smooth muscle cells (SMC). Everolimus, however, also blocks proliferation of endothelial cells and delays re-endothelization on the surface of a stented artery, thereby prolonging the healing process and possibly causing late restenosis and increasing the probability of thrombosis.

There is, therefore, a need for anti-restenosis agents that selectively block the proliferation of smooth muscle cells but allow for the proliferation of endothelial cells at a selected site.

### SUMMARY OF THE INVENTION

The present invention relates to an implantable medical device that includes a device body, an optional primer layer disposed over the device body, a reservoir layer disposed over the primer layer if opted, or over the device body, wherein the reservoir layer comprises one or more bioactive agents wherein at least one agent is an A1 adenosine receptor antagonist, an optional rate-controlling layer disposed over the reservoir layer and an optional topcoat layer disposed over the rate-controlling layer if opted, or over the reservoir layer. The device body can be a stent.

In various aspects, the A1 adenosine receptor antagonist can be 1,3-dipropyl-8-cyclopentylxanthine (DPCPX), cyclopentyltheophylline (CPT), 2-chloro-N(6)cyclopentyladenosine (CCPA), 8-cyclopentyl-1,3-dipropylxanthine (CPX), caffeine or theophylline.

In various aspects, the reservoir layer can include polyesters, poly(ether-esters), polyanhydrides, poly(L-lactide) (PLA), poly(D,L-lactide) (PDLLA), polyglycolide (PGA), polycaprolactone (PCL), polydioxanone (PDO), polytrimethylene carbonate (PTMC), and copolymers thereof.

Another aspect of the present invention relates to a method for treating or preventing a vascular disease that involves implanting a medical device according to the invention in a vessel of a patient in need thereof, wherein the one or more bioactive agents is present on the device in a therapeutically effective amount.

In various aspects, the vessel is a coronary artery in which case the vascular disease to be treated can include atherosclerosis, restenosis or vulnerable plaque.

In various aspects, the vessel is a peripheral artery in which case the vascular disease is peripheral arterial disease.

Another aspect of the present invention relates to a stent that includes a reservoir layer comprising polylactide and 1,3-dipropyl-8-cyclopentyl xanthine.

Another aspect of the present invention relates to a stent that includes a reservoir layer comprising polylactide-co-polyglycolide and 1,3-dipropyl-8-cyclopentyl xanthine or polylactide-co-procaprolactone and 1,3-dipropyl-8-cyclopentyl xanthine.

### DETAILED DESCRIPTION

The present invention provides an implantable medical device that includes a device body, an optional primer layer disposed over the device body, a reservoir layer disposed over the primer layer if opted, or over the device body, wherein the reservoir layer comprises one or more bioactive agents wherein at least one agent is an A1 adenosine receptor antagonist, an optional rate-controlling layer disposed over the reservoir layer and an optional topcoat layer disposed over the rate-controlling layer if opted, or over the reservoir layer.

As used herein, “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators, leads and electrodes for the preceding, implantable organ stimulators such as nerve, bladder, sphincter and diaphragm stimulators, cochlear implants, prostheses, vascular grafts, self-expandable stents, balloon-expandable stents, stent-grafts, grafts, PFO closure devices, arterial closure devices, artificial heart valves and cerebrospinal fluid shunts.

At present, preferred implantable medical devices for use with the invention are stents.

A stent refers generally to any device used to hold tissue in place in a patient's body. Particularly useful stents are those used for the maintenance of the patency of a vessel in a patient's body when the vessel is narrowed or closed due to diseases or disorders including, without limitation, tumors (in, for example, bile ducts, the esophagus or the trachea/bronchi), benign pancreatic disease, coronary artery disease, carotid artery disease, renal artery disease and peripheral arterial disease such as atherosclerosis, restenosis and vulnerable plaque. For example, a stent can be used to strengthen the wall of the vessel in the vicinity of a vulnerable plaque (VP). VP refers to a fatty build-up in an artery thought to be caused by inflammation. The VP is covered by a thin fibrous cap that can rupture leading to blood clot formation. Thus, a stent can not only maintain vessel patency but can act as a shield against VP rupture. A stent can be used in, without limitation, neuro, carotid, coronary, pulmonary, aortic, renal, biliary, iliac, femoral and popliteal as well as other peripheral vasculatures. A stent can be used in the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

In addition to the above uses, stents may also be employed for the localized delivery of therapeutic agents to specific treatment sites in a patient's body. Indeed, therapeutic agent delivery may be the sole purpose of the stent or the stent may be primarily intended for another use such as those discussed above with drug delivery providing an ancillary benefit.

A stent used for patency maintenance is usually delivered to the target site in a compressed state and then expanded to fit the vessel into which it has been inserted. Once at a target location, a stent may be self-expandable or balloon expandable. Due to the expansion of the stent, however, a stent coating must be flexible and capable of elongation.

Exemplary stent materials include, without limitation, stainless steel, nitinol, tantalum, tantalum alloy, titanium, titanium alloy, cobalt chromium, alloy x, niobium, niobium alloy, zirconium, zirconium alloy and biodegradable polymeric materials including for example, but not limited to, poly(L-lactide) (PLLA), poly(D,L-lactide) (PDLLA), PGA, PLLA-co-PGA (PLGA), PLLA-co-PCL (PLCL), PLLA-co-PTMC, PLLA-co-PDO, PLLA-co-PGA-co-PCL (PLGA-PCL), PLLA-co-PGA-co-PCL, poly(ether-esters) or polyanhydrides. Other suitable stent materials are known to those skilled in the art.

As used herein, “device body” refers to a fully formed implantable medical device with an outer surface to which no coating or layer of material different from that of which the device itself is manufactured has been applied. “Outer surface” means any surface, however spatially oriented, that is in contact with bodily tissue or fluids. An example of a “device body” is a BMS, i.e., a bare metal stent, which is a fully-formed usable stent that has not been coated with a layer of any material different from the metal of which it is made. It is to be understood that device body refers not only to BMSs but also to any uncoated device regardless of what it is made.

As used herein, “primer layer” refers to a coating consisting of a polymer or blend of polymers that exhibit good adhesion characteristics with regard to the material of which the device body is manufactured and good adhesion characteristics with regard to whatever material is to be coated on the device body. A primer layer is applied directly to a device body to serve as an intermediary layer between the device body and materials to be affixed to the device body. Examples of primers include, without limitation, silanes, titanates, zirconates, silicates, parylene, vinyl alcohol copolymers, acrylic acid copolymers, methacrylic acid copolymers, polyethyleneamine, polyallylamine, acrylate and methacrylate polymers with poly(n-butyl methacrylate), PLA and PLGA. Other suitable primer layer materials are known to those skilled in the art.

It is to be understood that an implantable medical device of the invention will necessarily include a reservoir layer but can optionally include a primer layer, a rate-controlling layer and a topcoat layer.

As used herein, “optional” means that the element modified by the term may or may not be present. For example, without limitation, a device body (db) that has coated on it an “optional” primer layer(pl), a drug reservoir layer (dr), an optional rate-controlling layer (rc) and an optional top-coat layer (tc) can refer to db+pi+dr+rc+tc, or db+pi+dr+rc, or db+pi+dr+tc, or db+pi+dr, or db+dr+tc, or db+dr+rc, or db+dr+rc+tc or db+dr.

As used herein, a material that is described as a layer “disposed over” an indicated substrate, e.g., a stent or another layer, refers to a relatively thin coating of the material applied directly to essentially the entire exposed surface of the indicated substrate. The term “disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, “reservoir layer” refers either to a layer of one or more bioactive agents applied to a medical device neat or to a layer of polymer or blend of polymers that has dispersed within its three-dimensional structure one or more bioactive agents. A polymeric drug reservoir layer is designed such that, without limitation, by elution or as the result of biodegradation of the polymer, the therapeutic substance is released from the layer into the surrounding environment.

The drug reservoir layer generally comprises a biocompatible polymer that can be hydrophobic or hydrophilic. Presently preferred reservoir layer polymers include polyesters, poly(ether-esters), polyanhydrides, PLA, PDLLA, PGA, PCL, PDO, PTMC, and copolymers thereof. Other suitable drug reservoir polymers are known to those skilled in the art.

As used herein, “biocompatible” refers to a polymer that both in its intact, as synthesized state and in its decomposed state, i.e., its degradation products, is not, or at least is minimally, toxic to living tissue; does not, or at least minimally and reparably, injure(s) living tissue; and/or does not, or at least minimally and/or controllably, cause(s) an immunological reaction in living tissue.

In various aspects, the reservoir layer contains one or more first bioactive agents wherein at least one is an A1 adenosine receptor antagonist.

As used herein, “antagonist” refers to a ligand that binds to a receptor but does not induce a biological response itself, rather blocks agonist-mediated responses.

The A1 adenosine receptor (A1R) has been shown to be specifically mitogenic to coronary SMC in vitro. Thus, A1R antagonists have a potential to selectively block SMC proliferation and restenosis, while not affecting re-endothelialization of the artery. One exemplary A1R antagonist is DPCPX, which is a signaling pathway specific drug that prevents proliferation of SMC by blocking A1Rs that are highly expressed in coronary SMC.

Presently preferred A1 adenosine receptor antagonists include, but are not limited to, DPCPX, CPT, CCPA, CPX, caffeine and theophylline. It is to be understood, however, that any A1 adenosine receptor antagonist, either presently known or that will become known in the future, will be suitable for coatings of the invention and as such are encompassed by the present invention. These antagonists will be readily ascertainable by those skilled in the art.

In addition to the A1R antagonist which must be present in the reservoir layer, additional A1R antagonists and/or other bioactive agents can also be present in the reservoir layer and suitable combinations will be easily ascertainable by those skilled in the art.

Other exemplary bioactive agents which can be present with the one or more A1R antagonists on the implantable medical device, also referred to herein as a drug or a therapeutic agent, include, without limitation, an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist or an antioxidant. Specific examples of the above agents are known to those skilled in the art.

As used herein, “rate-controlling layer” refers to a polymeric layer that is applied over a drug reservoir layer to modify a bioactive agent's rate of release into the environment. A rate-controlling layer may be used simply to “tune” the rate of release to exactly that desired by the practitioner or it may be a necessary adjunct to the construct because the polymer or blend of polymers with which the bioactive agent is compatible, with regard to coating as a drug reservoir layer, may be too permeable to the bioactive substance resulting in too rapid release and delivery of the bioactive substance into a patient's body.

Exemplary rate controlling layers include, but are not limited to, poly(ester amide)/2,2,6,6-tetramethylpiperidine-1-oxyl (PEA-TEMPO), polyanhydride, PLA, PGA, PLGA, PLCL, PLGA-PCL, PLGA-PEG or silk-elastin. Other suitable rate controlling layers are known to those skilled in the art.

As used herein, “top-coat layer” refers to an outermost layer that is in contact with the external environment and that is disposed as the final layer of a series of layers. The topcoat layer can be disposed over the rate-controlling layer of the invention, if opted, or over the reservoir layer.

Exemplary topcoat layers include, without limitation, PEG-PBT, poly(D,L-lactide), PLGA, PLGA-PEG, PLGA-PCL or silk-elastin. Other suitable topcoat layer materials are known to those skilled in the art.

A presently preferred aspect of the invention relates to a stent that comprises a reservoir layer that includes polylactide and 1,3-dipropyl-8-cyclopentyl xanthine.

Another presently preferred aspect of the present invention relates to a stent that includes a reservoir layer comprising polylactide-co-polyglycolide and 1,3-dipropyl-8-cyclopentyl xanthine or polylactide-co-procaprolactone and 1,3-dipropyl-8-cyclopentyl xanthine.

Another aspect of the present invention relates to a method for treating or preventing a vascular disease that involves implanting a medical device according to the invention in a vessel of a patient in need thereof, wherein the one or more bioactive agents is present on the device in a therapeutically effective amount. In various aspects, the vessel is a coronary artery in which case the vascular disease to be treated can include atherosclerosis, restenosis or vulnerable plaque. In other aspects, the vessel is a peripheral artery in which case the vascular disease is a peripheral arterial disease.

As used herein, “peripheral artery” refers to blood vessels outside of the heart and brain.

Methods of implanting medical devices are known to those skilled in the art.

As used herein, “treating” refers to the administration of a therapeutically effective amount of a bioactive agent to a patient known or suspected to be suffering from a vascular disease.

As used herein, a “therapeutically effective amount” refers to the amount of bioactive agent that has a beneficial effect, which may be curative or palliative, on the health and well-being of a patient with regard to a vascular disease with which the patient is known or suspected to be afflicted. A therapeutically effective amount may be administered as a single bolus, as intermittent bolus charges, as short, medium or long term sustained release formulations or as any combination of these.

As used herein, “known” to be afflicted with a vascular disease refers first to a condition that is relatively readily observable and or diagnosable. An example, without limitation, of such a disease is atherosclerosis, which is a discrete narrowing of a patient's arteries. Restenosis, on the other hand, while in its latter stages, like atherosclerosis, is relatively readily diagnosable or directly observable, may not be so in its nascent stage. Thus, a patient may be “suspected” of being afflicted or of being susceptible to affliction with restenosis at some time subsequent to a surgical procedure to treat an atherosclerotic lesion. Further, while restenosis tends generally to occur at the same locus as a previous atherosclerotic lesion, it may not be exactly so, so a region of a segment of a vessel somewhat distant from the site of the initial atherosclerosis may in fact be the site of restenosis.

As used herein, a “vascular disease locale” refers to the location within a patient's body where an atherosclerotic lesion(s) is present, where restenosis may develop, the site of vulnerable plaque(s) or the site of a peripheral arterial disease.

An atherosclerotic lesion refers to a deposit of fatty substances, cholesterol, cellular waste products, calcium and/or fibrin on the inner lining or intima of an artery.

Restenosis refers to the re-narrowing or blockage of an artery at or near the site where angioplasty or another surgical or interventional procedure was previously performed to remove a stenosis.

Vulnerable plaque on the other hand is quite different from either atherosclerosis or restenosis and would generally come under the designation “suspected” affliction. This is because vulnerable plaque occurs primarily within the wall of a vessel and does not cause prominent protrusions into the lumen of the vessel. It is often not until it is “too late,” i.e., until after a vulnerable plaque has broken and released its components into the vessel, that its presence is even known. Numerous methods have and are being investigated for the early diagnosis of vulnerable plaque but to date none have proven completely successful. Thus, the regional treatment of a segment of a vessel suspected of being afflicted with vulnerable plaque may be the best way to address such lesions.

As used herein, “peripheral arterial disease” refers to a condition similar to coronary artery disease and carotid artery disease in which fatty deposits build up in the inner linings of the artery walls thereby restricting blood circulation, mainly in arteries leading to the kidneys, stomach, arms, legs and feet.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. An implantable medical device comprising:
a device body;
an optional primer layer disposed over the device body;
a reservoir layer disposed over the primer layer if opted, or over the device body, wherein the reservoir layer comprises one or more bioactive agents wherein at least one agent is an A1 adenosine receptor antagonist;
an optional rate-controlling layer disposed over the reservoir layer; and
an optional topcoat layer disposed over the rate-controlling layer if opted, or over the reservoir layer.

2. The implantable medical device according to claim 1, wherein the A1 adenosine receptor antagonist comprises 1,3-dipropyl-8-cyclopentylxanthine, cyclopentyltheophylline, 2-chloro-N(6)cyclopentyladenosine, 8-cyclopentyl-1,3-dipropylxanthine, caffeine and theophylline.

3. The implantable medical device according to claim 1, wherein the reservoir layer comprises polyesters, poly(ether-esters), polyanhydrides, poly(L-lactide), poly(D,L-lactide), polyglycolide, polycaprolactone, polydioxanone, polytrimethylene carbonate, and copolymers thereof.

4. The implantable medical device according to claim 1, wherein the device body comprises a stent.

5. A method for treating or preventing a vascular disease comprising implanting the medical device according to claim 1 in a vessel of a patient in need thereof, wherein the one or more bioactive agents is present on the device in a therapeutically effective amount.

6. The method according to claim 5, wherein the vessel is a coronary artery.

7. The method according to claim 6, wherein the vascular disease is atherosclerosis, restenosis or vulnerable plaque.

8. The method according claim 5, wherein the vessel is a peripheral artery.

9. The method according to claim 8, wherein the vascular disease is peripheral arterial disease.

10. A stent comprising:
a reservoir layer comprising polylactide and 1,3-dipropyl-8-cyclopentyl xanthine.

11. A stent comprising:
a reservoir layer comprising polylactide-co-polyglycolide and 1,3-dipropyl-8-cyclopentyl xanthine or polylactide-co-procaprolactone and 1,3-dipropyl-8-cyclopentyl xanthine.
